(19) United States
(12) Patent Application Publication
(10) Pub. No.: US 2023/0041085 A1
(43) Pub. Date: Feb. 9, 2023

(54) OXYGEN REDUCTION REACTIONS AND INTERBRAIN SYNCHRONY

(71) Applicant: Joel Steven Goldberg, Hillsborough, NC (US)

(72) Inventor: Joel Steven Goldberg, Hillsborough, NC (US)

(21) Appl. No.: 17/958,328

(22) Filed: Oct. 1, 2022

(51) Int. Cl. A61N 2/00; A61N 2/02

(52) U.S. Cl. CPC A61N 2/006; A61N 2/02

(57) ABSTRACT

Hyperscanning, the simultaneous measurement of brain activity among dyads or groups, has shown synchrony measured by fMRI, EEG, MEG, and fNIRS. This synchrony is evident during cooperative decision-making, but the underlying mechanism has not been elucidated. At minimum, in order for synchrony to occur, systems must oscillate and share information. Through the skin effect, changing magnetic fields permeate the human central nervous system, can share information from dyads and groups, and can synchronize oscillating oxygen reduction reactions (ORR) in the mitochondria. In vitro evidence has shown that ORR can be influenced μ-m tesla, extremely low frequency magnetic fields without producing thermal or ionization effects. Ascertaining the unique characteristics of such magnetic fields that can produce interbrain synchrony is a daunting task; however, this discovery could benefit impasse conflict negotiations.

### CROSS-REFERENCES TO RELATED APPLICATIONS

None

### FEDERALLY FUNDED RESEARCH

Not applicable

### BACKGROUND OF THE INVENTION

### Synchronization of Systems

Humans are happy when they are in synchrony with others. This occurs in team sports, choral singing, and in love.[1, 2] The etiology of this synchrony has been elusive for many years, but it also occurs in pacemaker cells, fireflies, cycling of menstrual periods, and walking in tandem. It is well known that many oscillating systems have a natural tendency to synchronize, and synchronization can occur in coupled inherent oscillators and/or coupled movements in space, such as swarms of fishes or birds in flight.[3]

In 1665, Christopher Huygens first observed and reported the synchronization of pendulum clocks attached to a common structure. (FIG. 1)

Huygens wrote “ . . . It is quite worth noting that when we suspended two clocks so constructed from two hooks embedded in the same wooden beam, the motions of each pendulum in different swings were so much in agreement that they never receded the least bit from each other, and the sound of each was always heard simultaneously. Further, if this agreement was disturbed by some interference, it re-established itself in a short time. For a long time I was amazed at this unexpected result, but after a careful examination finally found that the cause of this is due to the motion of the beam, even though this is hardly perceptible.”

Huygens noted that the clocks synchronized in opposite directions (180 degrees out of phase), and he referred to this observation as an “odd kind of sympathy”. If the clocks did not share information via a common attachment, no synchronization occurred.

It is now recognized that synchronization of periodic, chaotic, or probably stochastic systems can occur when the systems oscillate and can share information—the two fundamental properties of systems required for synchronization. There are many types of synchrony, and the two types most relevant to this invention are reciprocal and induced. (FIG. 2)[4] Reciprocal synchrony occurs between two systems without external forces, and induced synchrony occurs between two systems modulated by an external force.

In 1997, Pecora et al. explained the synchronization of chaotic systems.[5] Since that time, it has been shown that two chaotic systems, two Chua's circuits, the simplest electronic chaotic system, can synchronize without physical contact presumably through magnetic induction.[6] It has also been shown that a chaotic system modeled by the Belouvsa-Zhabotinsky reaction could be influenced by low frequency changing electromagnetic fields.[7]

Hyperscanning

In vivo, hyperscanning, the simultaneous measurement of brain activity has demonstrated synchrony or coherence in dyads or groups in many activities including, performing music, didactic instruction, interaction of lovers, and cooperative decision-making the last of which is the focus of this invention. There are skeptics of hyperscanning, which was discovered in 2002 by Montague, but the sheer number of publications by a multitude of investigators from laboratories across the globe who have reported on the observation lends credence to its existence.[4, 8, 9] Table 1 is a timetable of techniques used in hyperscanning.

TABLE 1
Timetable of hyperscanning studies

| Year | Study | Method | Investigator |
|---|---|---|---|
| 2002 | First observation | fMRI | Montague et al.[8] |
| 2007 | Prisoner's dilemma | EEG | Babiloni et al.[10] |
| 2011 | Music | EEG | Babiloni et al.[11] |
| 2011 | Tapping synchronization | fNIRS | Funane et al.[12] |
| 2012 | Auditory interaction | MEG | Baess et al.[13] |
| 2017 | Classroom instruction | EEG | Dikker et al.[14] |
| 2019 | Eye contact | fMRI | Koike et al.[15] |

The etiology of these observations has not been explained, but this invention explains the possible neurochemistry of synchronous hyperscanning and implications for conflict resolution.

Oxygen Reduction Reactions, Extremely Low Frequency Magnetic Fields, and Hyperscanning

Oxygen reduction reactions (ORR) that occur in the mitochondria are some of the most important reactions in nearly all life. These reactions couple oxygen reduction to adenosine triphosphate (ATP) production, the major currency for energy, and without these reactions ATP production cannot proceed through electron transport and oxidative phosphorylation. The mechanisms of ORR are complicated, but two major pathways are described:

O₂+4H⁺+4e⁻↔H₂O 1.

O₂+2H⁺+2e⁻↔H₂O₂ 2.

H₂O₂+2H⁺+2e⁻↔2H₂O

In the mitochondria, the more efficient four electron pathway occurs at cytochrome C in the mitochondrial matrix. (FIG. 3, Label 1) Uncoupling of this pathway with cyanide is nearly always fatal. Since this pathway is so vital for basal ATP production, it is not surprising that a second or redundant pathway exists. (FIG. 3, Label 2) This pathway also occurs in the mitochondrial matrix with single electron reductions in complexes I and III, and a branch of this pathway produces H₂O₂, which is reduced by two electrons with catalase to 2H₂O. (FIG. 3, Label 3) [16] This branch pathway is autocatalytic since O₂ is both a reactant and product; thus, it is presumed that concentrations of products and reactants oscillate along with free energy. The less efficient pathway is known to produce reactive oxygen species (ROS) including superoxide, (O2⁻), hydrogen peroxide (H₂O₂), and hydroxyl radical (.OH).[17]

Two unpaired electrons in the outer shell of O₂ are responsible for the paramagnetic properties of oxygen gas, and elegant work in air fuel cells with platinum cathodes has demonstrated that oxygen reduction rates can be increased with a static magnetic field (0.56 T and 1.42 T) in the vicinity of the cathode. [18, 19] Furthermore, it has been demonstrated that an approximately 80 mT magnetic field with a low frequency of approximately 280 Hz using an Oncomagnetic Device, altered mitochondrial oxidative processes in mitochondria of cancer cells and plant cells.[20] The magnetic field strength of the earth is approximately 25-65 μT.

Nearly all experimental investigations of non-thermal, non-ionizing magnetic field effects in biological systems have focused on radical pair mechanisms (RPM). Unpaired electrons in the presence of magnetic fields can undergo Zeeman splitting and/or hyperfine interactions which can produce triplet energy levels from singlet energy levels. Interacting radical pairs that have been transformed can change the kinetics and products of the RPM.[21] A commonly studied free radical reaction is: FAD.⁻+TrpH.⁻=[FAD.⁻+TrpH.⁻] that occurs in cryptochrome flavoproteins which have been activated by blue light. This reaction has been found in numerous organisms, and it may be the explanation for an in vivo magnetic compass and circadian rhythms. In addition to direct paramagnetic effects on ORR, evidence suggests that magnetic fields can exert their influence on ORR via RPM.

A plethora of inconclusive literature describing non-thermal and non-ionizing effects of electromagnetic radiation on human nervous system function exists. These include studies from extremely low frequency (ELF) to gigahertz frequencies, various wave conformations, and a multitude of waveform amplitudes. The most convincing observations support ELF effects from magnetic field intensities in the mT range. [22, 23] This effect is a magneto chemical effect that is presumed to occur regardless of genotype or phenotype.

The brain receives information from the senses which is processed at multiple locations, and if an action is required, the motor cortex is activated. Reward centers of the brain, mostly comprised of dopaminergic neurons, often but not exclusively, determine whether actions are initiated. Humans communicate their actions with the outside world with movements that include speech, writing, and gestures. Therefore, the motor cortex is the structure that inevitably determines human action. The energy required for motor movement in most instances is significantly greater than for sensory or autonomic function, and thus ORR that regulate ATP synthesis are intimately coupled to human action.[24]

It is proposed that some hyperscanning effects where participants are screened from common sensory stimuli such as light, sound, or touch is caused by coupling of ORR with magnetic fields. Finding the optimum amplitude, frequency, and conformation of electromagnetic radiation that couples mitochondrial ORR and produces synchrony in dyads or groups is a daunting task, but the benefits, particularly in understanding brain activity in cooperative decision-making, are potentially so enormous that extraordinary resources should be allocated, especially if induced synchrony is possible.

### ORR and Anesthesia

Oxygen reduction reactions in the brain may explain volatile anesthetic effects and inhalation of some solvents, since any of these agents will decrease the partial pressure of oxygen and presumably decrease ATP production in the mitochondrial matrix. In fact, the minimum alveolar concentration of halothane (MAC) is decreased during hypoxia which would be the expected observation if the partial pressure of oxygen in the mitochondria is reduced, decreasing ATP production.[25, 26]

### Oxidation ORR and Ischemia

Another clinical observation supports the importance of partial pressure of oxygen and ORR in the mitochondria. Ischemia increases the partial pressure of nitrogen in tissues, and subsequently decreases the partial pressure of oxygen in the mitochondrial matrix. It is unlikely that a vacuum forms in the tissues when oxygen is consumed. Therefore, it may be essential to denitrogenate ischemic tissues, along with restoring oxygen tension, to treat ischemia, especially in the brain and heart. [27]

### DRAWINGS

FIG. 1 was drawn by Huygens in 1665 and shows pendulum clock anti-synchronization.

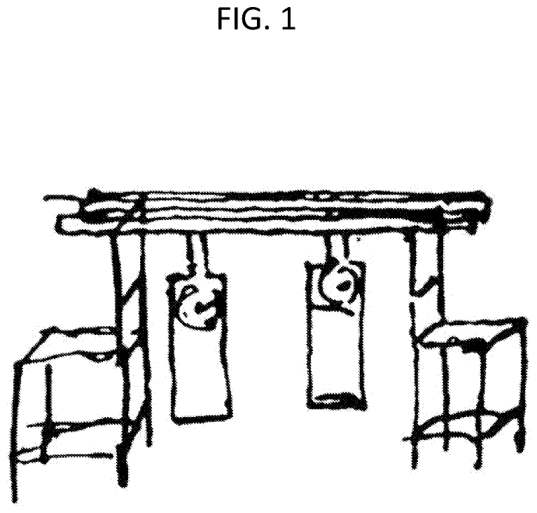

FIG. 2 shows different types of synchrony in simple pendulum clocks.

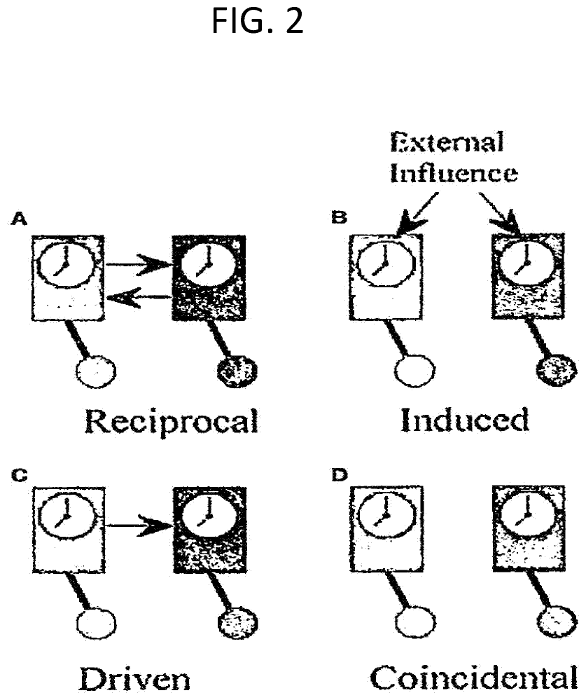

FIG. 3 shows a mitochondrion. Label 1 shows the four electron transfer ORR. Label 2 shows the single electron reduction reactions. Label 3 shows the branched pathway associated with autocatalytic ORR.

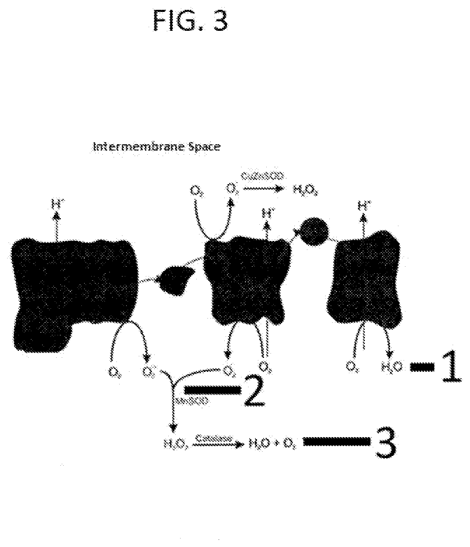

FIG. 4 shows a skull. Label 1 is the area of the pterions.

### DETAILED DESCRIPTION OF THE INVENTION

Oxygen reduction reactions are susceptible to μ-mT low frequency magnetic fields, and these fundamental reactions supply energy in the form of ATP to humans during basal metabolism. Low intensity μ-mT low frequency 50-300 Hz magnetic fields can change the kinetics of ORR without thermal or ionizing effects, and these fields can provide shared information between dyads and groups. This shared information via magnetic fields can produce interbrain synchrony under certain conditions. In nature, synchrony is commonly observed in fireflies, cyclic menstrual periods, walking in tandem, and cooperative decision-making. The latter situation applies to resolution of human conflict, and it is proposed that information through artificially produced changing magnetic fields of an optimum intensity, frequency, and conformation can promote cooperative decision-making.

Theoretical Calculations

Electromagnetic energy can interact with the human nervous system either via the senses or directly through the skull. In the latter case, the skin effect predicts the penetration (skin depth) of an electromagnetic wave in a human body which is a conductor.

δ=[p/(π*f*μ₀*μᵣ)]^{1/2}

### Where:

### δ—Skin Depth

p—Resistivity of the tissues
f—Frequency of the electromagnetic wave
μ₀=4π*10⁻⁷ H/m—Permeability of free space
μᵣ=—Relative magnetic permeability of tissues

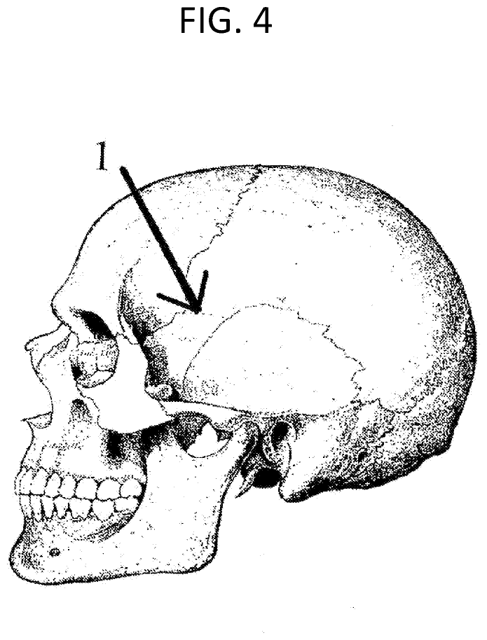

The skin effect also predicts that low frequency (f) will favor greater penetrations, which is consistent with experimental findings. Previously reported frequencies to generate phosphenes (visual cortex stimulation without light stimulation of the retina) were 10-35 Hz.[23] From the skin to the brain, electromagnetic waves will attenuate, so the thinnest area of skin to brain depth, which lies over the pterions, will favor penetration. (FIG. 4, Label 1)

Beneath the pterions lie the temporal bone and amygdala. There is good evidence that the amygdala directly communicates with prefrontal motor cortex.[28] Thus, electromagnetic waves that enter the brain may have a direct route to motor actions and influence human behavior. The electromagnetic force per unit volume on paramagnetic oxygen gas is:[19]

Fₘ=μ₀*χ*H*(dH/dy)

### Where:

μ₀=4π*10⁻⁷ H/m— Permeability of free space
χ=volume magnetic susceptibility for oxygen gas (1.91×10⁻⁶)
H=magnetic field strength
(dH/dy)=magnetic field gradient
The paramagnetic force will be greatest when (dH/dy) is greatest, so a square wave will produce a greater force than a sine wave with all other parameters unchanged.

Therefore, based on the synthesis of multiple experiments describing biological effects of non-thermal and non-ionizing electromagnetic radiation, it is believed that the most likely electromagnetic energy that could couple with ORR will include the following characteristics:

A. Frequency: 1-300 hertz

B. Intensity: μ-m tesla

C. Conformation: square wave

### Alternative Explanations

While synchronization of ORR by changing magnetic fields may be a plausible explanation for hyperscanning, there may be other possibilities and possible flaws in this concept. Human brain activity can be coupled to dyads or groups through the senses. Hearing music, viewing objects, and touch are common experiences that produce synchronization. Human activity in a concert often includes synchronous hand clapping. Group viewing of art in a museum and intimate touch between lovers seem synchronized. Although these sensations link human action, the experimental conditions in many hyperscanning experiments control for environmental influences on brain activity. However, these studies do not exclude ambient magnetic fields which are mutually shared by dyads and groups. In addition, there are numerous biochemical pathways in the brain such as glycolysis which are known to oscillate, but these pathways do not include paramagnetic oxygen and/or RPM.

### Benefits to Society

The concepts presented in this invention are broad and speculative, yet extrapolated from current scientific observations and knowledge. If such intensities, frequencies, and conformations of magnetic fields can be discovered to aid conflict negotiations among dyads and groups, it would be an immense benefit to the negotiation process, especially where human lives are at risk. If such magnetic fields are discovered, the possibility that billions of humans could become “in sync” via Internet connections could become a reality.

Nikola Tesla purportedly commented: “Alpha waves in the human brain are between 6 and 8 hertz. The wave frequency of the human cavity resonates between 6 and 8 hertz. All biological systems operate in the same frequency range. The human brain's alpha waves function in this range and the electrical resonance of the earth is between 6 and 8 hertz. Thus, our entire biological system—the brain and the earth itself—work on the same frequencies. If we can control that resonate system electronically, we can directly control the entire mental system of humankind”. However, this invention is not mind control. This invention describes a novel method to improve impasse conflict negotiations, especially during multi-track diplomacy, utilizing observations from human neurophysiology.

### REFERENCES

1. Pan, Y., et al., Cooperation in lovers: An fNIRS-based hyperscanning study. Hum Brain Ma pp, 2017. 38(2): p. 831-841.
2. Kinreich, S., et al., Brain-to-Brain Synchrony during Naturalistic Social Interactions. Sci Rep, 2017. 7(1): p. 17060.
3. O'Keeffe, K. P., H. Hong, and S. H. Strogatz, Oscillators that sync and swarm. Nat Commun, 2017. 8(1): p. 1504.
4. Burgess, A. P., On the interpretation of synchronization in EEG hyperscanning studies: a cautionary note. Front Hum Neurosci, 2013. 7: p. 881.
5. Pecora, L. M., et al., Fundamentals of synchronization in chaotic systems, concepts, and applications. Chaos, 1997. 7(4): p. 520-543.
6. Goldberg, J. S., Jackson, C. L., METHOD TO IMPROVE OUTCOMES DURING NEGOTIATIONS US 2020/0108264 A1
7. Goldberg, J. S., THERMODYNAMIC MODEL OF A NERVOUS SYSTEM US 2015/0379898 A1.
8. Montague, P. R., et al., Hyperscanning: simultaneous fMRI during linked social interactions. Neuroimage, 2002. 16(4): p. 1159-64.
9. Nam, C. S., et al., Direct Communication Between Brains: A Systematic PRISMA Review of Brain-To-Brain Interface. Front Neurorobot, 2021. 15: p. 656943.
10. Babiloni, F., et al., Cortical activity and connectivity of human brain during the prisoner's dilemma: an EEG hyperscanning study. Annu Int Conf IEEE Eng Med Biol Soc, 2007. 2007: p. 4953-6.
11. Babiloni, C., et al., Brains “in concert”: frontal oscillatory alpha rhythms and empathy in professional musicians. Neuroimage, 2012. 60(1): p. 105-16.
12. Funane, T., et al., Synchronous activity of two people's prefrontal cortices during a cooperative task measured by simultaneous near-infrared spectroscopy. J Biomed Opt, 2011. 16(7): p. 077011.
13. Baess, P., et al., MEG dual scanning: a procedure to study real-time auditory interaction between two persons. Front Hum Neurosci, 2012. 6: p. 83.
14. Dikker, S., et al., Brain-to-Brain Synchrony Tracks Real-World Dynamic Group Interactions in the Classroom. Curr Biol, 2017. 27(9): p. 1375-1380.
15. Koike, T., et al., What Makes Eye Contact Special? Neural Substrates of On-Line Mutual Eye-Gaze: A Hyperscanning fMRI Study. eNeuro, 2019. 6(1).
16. Kowaltowski, A. J., et al., Mitochondria and reactive oxygen species. Free Radic Biol Med, 2009. 47(4): p. 333-43.
17. Hernansanz-Agustin, P. and J. A. Enriquez, Generation of Reactive Oxygen Species by Mitochondria. Antioxidants (Basel), 2021. 10(3).
18. Okada, T., Wakayama, N. I., Wang, L., et al, The effect of magnetic field on the oxygen reduction reaction and its application in polymer electrolyte fuel cells. Electrochimica Acta, 2003. 48: p. 531-539.
19. Wakayama, N. I., Okada, T., Okano, J., Ozawa, T., Magnetic Promotion of Oxygen Reduction Reaction with Pt Catalyst in Sulfuric Acid Solutions. Jpn. J. Appl. Phys., 2001. 40: p. L269-L271.
20. Sharpe, M. A., et al., Rotating Magnetic Fields Inhibit Mitochondrial Respiration, Promote Oxidative Stress and Produce Loss of Mitochondrial Integrity in Cancer Cells. Front Oncol, 2021. 11: p. 768758.
21. Castello, P., P. Jimenez, and C. F. Martino, The Role of Pulsed Electromagnetic Fields on the Radical Pair Mechanism. Bioelectromagnetics, 2021. 42(6): p. 491-500.
22. Cook, C. M., A. W. Thomas, and F. S. Prato, Human electrophysiological and cognitive effects of exposure to ELF magnetic and ELF modulated RF and microwave fields: a review of recent studies. Bioelectromagnetics, 2002. 23(2): p. 144-57.
23. Goldberg, J. S., METHOD TO OPTIMIZE ELECTRODE PLACEMENT FOR CRANIAL ELECTRICAL STIMULATION US 2016/0129238 A1.
24. Goldberg, J. S., TREAMENT OF AMYOTROPHIC LATERAL SCLEROSIS WITH LACTATE US 2016/0271085 A1.
25. Cullen, D. J., et al., The effects of hypoxia and isovolemic anemia on the halothane requirement (MAC) of dogs. II. The effects of acute hypoxia on halothane requirement and cerebral-surface Po2, Pco2, pH and HCO3. Anesthesiology, 1970. 32(1): p. 35-45.
26. Cullen, D. J. and E. I. Eger, 2nd, The effects of hypoxia and isovolemic anemia on the halothane requirement (MAC) of dogs. 3. The effects of acute isovolemic anemia. Anesthesiology, 1970. 32(1): p. 46-50.
27. Goldberg, J. S., METHOD TO DECREASE BRAIN INJURY FOLLOWING CEREBRAL ISCHEMIA US 2016/0228664 A1.
28. Grezes, J., et al., A direct amygdala-motor pathway for emotional displays to influence action: A diffusion tensor imaging study. Hum Brain Mapp, 2014. 35(12): p. 5974-83.

Having described my invention, I claim:

1. A method to enhance synchrony in a brain of a human utilizing an extremely low frequency, low intensity, square wave magnetic field such that the field modulates an oxygen reduction reaction in a human brain mitochondrion.

2. A method to enhance synchrony in a brain of a human utilizing a 1-300 hertz, and 1-100 millitesla, square wave magnetic field such that the field modulates an oxygen reduction reaction in a human brain mitochondrion.
